(19) United States
(12) Patent Application Publication
(10) Pub. No.: US 2005/0256425 A1
(43) Pub. Date: Nov. 17, 2005

(54) Device, system and method for extracting and preparing brain tissue

(75) Inventor: Stanley B. Prusiner, San Francisco, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: InPro Biotechnology, Inc.

(21) Appl. No.: 10/843,863

(22) Filed: May 11, 2004

(51) Int. Cl. A61B 17/32; A61B 17/14; A61B 10/00

(52) U.S. Cl. 600/567; 606/184

(57) ABSTRACT

A syringe type tissue extraction device is disclosed. The device comprises a sharp, cylindrical metal cuff at one end of a transparent tube which is pushed into brain tissue. Once inserted a plunger is pulled back and by observation through the transparent tube a determined amount of tissue is extracted. The device is then used to place the sample in a single homogenization tube or to punch through a seal above a well of a 96 well plate and the tissue in the tube is inserted into the well. Each well (or the single tube) of the plate holds beads and once tissue is inserted in all the wells the plate is shaken to homogenize the tissue in each well in order to prepare the sample for assaying.

### FIELD OF THE INVENTION

This invention relates to the field of devices for the removal of tissue and particularly brain tissue and a methods of assaying such.

### BACKGROUND OF THE INVENTION

A range of different biopsy devices utilizing needle aspiration to extract tissue samples are known to those skilled in the art. Such devices are useful to obtain samples of tissue, such as tissue suspected of being cancerous. The tissue samples may be examined in order to confirm such suspected diagnosis. Such devices are frequently used when sampling suspected cancerous tissue in the lungs, liver, adrenal glands, kidneys, breasts, and prostate.

Biopsy devices may extract a sample of tissue from a tissue mass by either drawing a tissue sample into a hollow needle via an external vacuum force or by severing and containing a tissue sample within a notch formed in a stylet. Typical of such devices utilizing a vacuum force are U.S. Pat. No. 5,246,011 issued to Cailouette and U.S. Pat. No. 5,183,052 issued to Terwilliger. Such devices contemplate advancing a hollow needle into a tissue mass and applying a vacuum force to draw a sample into the needle and hold the same therein while the tissue is extracted.

Biopsy devices may utilize a biopsy needle with a tissue sample recovery notch formed thereon to extract a specimen, such as described in U.S. Pat. No. 3,477,423 issued to Griffith, often referred to as the TRU-CUT needle and U.S. Pat. No. 4,776,423 issued to Beraha et al.

Specially designed cannulas may be used to enhance the cutting and recovery of tissue samples, as well as combining the application of a vacuum force to draw a tissue sample into a biopsy cavity prior to cutting the tissue contained therein. U.S. Pat. No. 4,708,147 issued to Haaga discloses a cannula for a biopsy needle designed to cut a sample of tissue and then applying a vacuum to the cannula such that the tissue is drawn into the cannula and thus retained therein for recovery. Additionally, U.S. Pat. No. 3,844,272 issued to Banko discloses a biopsy device wherein a suction force, created by a vacuum, draws a sample of tissue into a receiving compartment whereby two coaxial members are rotated relative to each other so that the members essentially coact to cut off the specimen and place it into a compartment.

Many of these devices are complex and expensive to produce and are often reused which requires sterilization of the device after each use. Accordingly, there exists a substantial need in the art to provide a tissue sample extractor capable of effectively and efficiently drawing in a suitable sample of tissue and isolating the tissue sample within the biopsy device. Additionally, there is a need for a biopsy device that is easy to use and can effectively be manipulated by one hand. Furthermore, there is a need in the art to provide a tissue sample extractor that not only provides tissue samples of sufficient size, but allows the sample to be readily placed in a container for treatment and later analysis. Further, it would be of even greater advantage to provide a tissue sample extractor having the above-mentioned features which is also a completely disposable device which can be easily and cheaply manufactured.

### SUMMARY OF THE INVENTION

A device, system and method for the extraction of brain tissue and its preparation for being subjected to an assay is disclosed. The device is comprised of transparent graduated hollow tube having a tubular metal cuff positioned into one end and a flange or protruding rim at the opposite end. A plunger comprised of a rubber or polymeric material is connected to a first end of a shaft which is positioned inside the hollow tube such that it can be moved to create suction. The shaft may have a flange at its second end allowing for the plunger to be pulled through the tube and create suction. The tubular metal cuff is connected to the hollow tube at its first end. The second or distal end of the cuff has a sharp edge which can be easily forced into brain tissue to cut out a piece of about 0.1 to 10 ml, 0.1 to 2 ml, or 0.1 to 1.0 ml in volume which tissue is pulled into the tube with the aid of the plunger.

An aspect of the invention is a method comprising the steps of:
(a) inserting into a brain of a farm animal a tissue extraction device comprising a graduated, cylindrical, transparent tube comprising a first end and a second end, a metal cuff inserted into the first end of the tube and extending outward from the first end, the cuff comprising a circular metal cylinder comprising a sharpened end extending from the first end of the tube and a plunger comprised of a shaft connected to a stopper at a first end and a flange at a second end;
(b) withdrawing the plunger to draw about 0.3 milliliter of brain tissue into the tube; and
(c) moving the plunger forward to expel the brain tissue from the tube into a well of a 96 well plate comprised of 96 interconnected wells having a continuous sealant cover over openings of all of the wells, wherein the internal diameter of each well of the 96 well plate is substantially the same as the external diameter of the cylindrical, transparent tube of the extraction device which tube has therein a plurality of beads.

Yet another aspect of the invention is carried out by repeating steps (a), (b) and (c) a plurality of times wherein a new tissue extraction device is used in each set of repeated steps to extract tissue from another animal which is expelled into a different well.

In accordance with the above method in step (c) the brain sample may be expelled into an individual tube such as a tube having a volume of about 1.5 to 3 or more particularly about 2 ml in volume which is preferably a polypropylene tube containing beads which can be shaken to homogenize the brain. Such a method can be used in accordance with a low through-put system which does not require obtaining large numbers of samples which would be used with the high through-put 96 well plate system.

Once the tissue is extracted it is extruded from the tube by forcing the plunger down. The extruded tissue is placed in a well of a 96 well plate covered with a breakable foil liner. The well contains beads which aid in homogenizing the tissue sample placed therein.

An aspect of the invention is that the tissue extraction device is simple and easy to use for the rapid extraction of tissue.

Another aspect of the invention is that the tissue can be easily and rapidly extruded from the device into the next available well for later treatment.

Yet another aspect of the device is that it is inexpensive and as such can be disposed of after a single use.

These and other objects, advantages, and features of the invention will become apparent to those persons skilled in the art upon reading the details of the device, system and method as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 is a cross-sectional view of an embodiment of a tissue extraction device of the invention;

FIG. 2 is a cross-sectional view of another embodiment of a tissue extraction device of the invention with a biasing means;

FIG. 3 is a cross-sectional view of yet another embodiment of a tissue extraction device of the invention;

FIG. 4 is a cross-sectional view of still another embodiment of a tissue extraction device of the invention;

FIG. 5 is a perspective view of the 96 well plate component of the invention;

FIG. 6 is a photo showing a cow brainstem and pointing out the OBEX region;

FIG. 7 is a photo of a cow brainstem in the hand of a gloved researcher with a device of the invention being positioned thereon; and

FIG. 8 is a photo of a cow brainstem being held while a device of the invention is being used.

### DETAILED DESCRIPTION OF THE INVENTION

Before the present device, system and method is described, it is to be understood that this invention is not limited to particular embodiment described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a sample” includes a plurality of such samples and reference to “the assay” includes reference to one or more assays and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Tissue Extraction Device

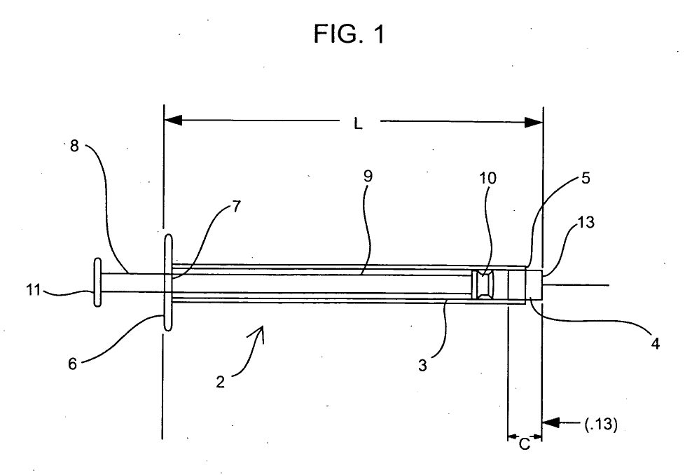

Referring now to FIG. 1 showing a schematic cross-sectional view of the device 2. The device 2 is comprised of a transparent tube 3 comprised of a cuff 4 at a first end 5 and a flange 6 at its second end 7.

A plunger 8 is comprised of a shaft 9 with a rubber or polymeric stopper 10 inserted inside the tube 3. The shaft 9 has a flange 11 which can be easily pulled to move the stopper 10 toward the end 7 of the tube 3 and create a vacuum inside the tube 3.

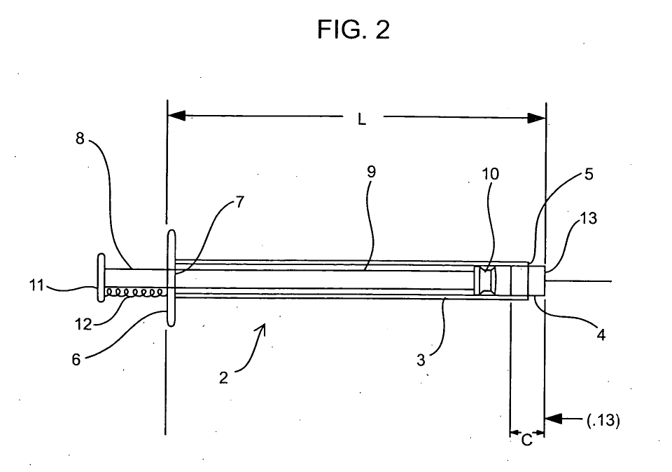

In one embodiment of the device as shown in FIG. 2 a biasing means such as spring 12 is positioned on the device such as between the flange 7 and the flange 11. The biasing means 12 can be used to bias the flange 11 toward or away from the flange 7 thereby moving the stopper 10 within the tube 3. In one embodiment the biasing means 12 is designed such that when the user forces the flange 11 forward to the maximum extent the stopper 10 is forced all the way forward in the tube 3. At this point the cuff 4 is used to cut into the tissue and the tube 3 may also be forced into the tissue. Alternatively or in combination therewith the pressure on the flange 11 is released and the biasing means 12 forces the flange 11 away from the flange 7 by a predetermined amount. This predetermined amount causes the stopper 10 to move a predetermined length thereby causing a predetermined volume of tissue (e.g. 1 ml±5%) to be drawn into the tube 3.

In the embodiment shown within FIG. 1 the cuff 4 has an external diameter which is substantially the same as the internal diameter of the tube 3. Accordingly, the cuff 4 fits snugly into the tube 3 and may be held in position by the snugness of the fit between the internal diameter of the tube 3 and the external diameter of the cuff 4 alone or in combination with an adhesive between the outside of the cuff 4 and the inside of the tube 3. In the embodiment shown (FIGS. 1 and 2) the external diameter of the tube 3 and the external diameter of the cuff 4 are different with the tube 3 having a larger diameter.

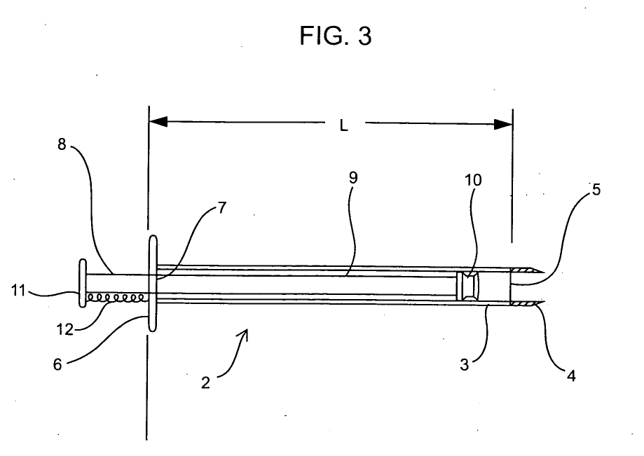

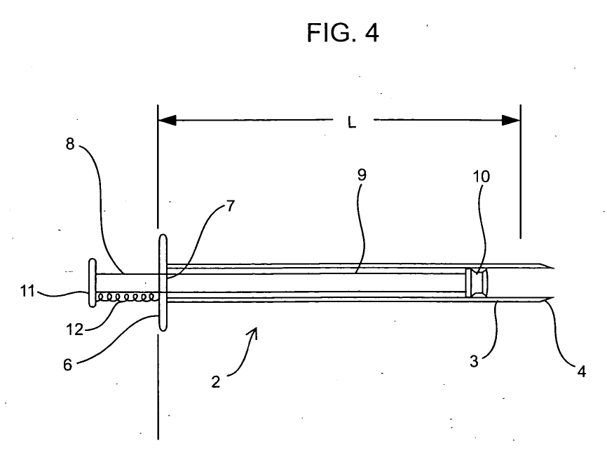

However, in the embodiment of FIG. 3 for the internal and external diameter of the cuff 4 matches the internal and external diameter of the tube 3. When the external diameters of both the tube 3 and cuff 4 match each other the device can be more easily inserted into tissue without any interruption caused by the end 5 of the large diameter tube 3. In yet another embodiment (FIG. 4) the cuff 4 may become an integral part of the tube 3 essentially becoming a sharpened end of the tube 3.

Making the cuff 4 integral with the tube 3 is, as described above and shown in FIG. 4, a possible embodiment. However, the device 2 can be simply manufactured from currently available syringe devices which are cut and modified by the addition of a cuff 4. This simplifies the manufacture and reduces the cost of the devices and thus reduces the cost of the entire procedure in which the devices are used. When a standard syringe is used the syringe is comprised of a transparent polymeric material which syringe holds approximately 1 ml of volume (±5% or ±1% or ±0.1%) and includes graduation markings on its side to indicate when the 1 ml volume is reached and may also have various fractional points indicated thereon such as ½, ¼, ⅕, ⅓, or 1/10 markings.

Although the device of the invention could be used for the extraction of various types of tissue it is preferably used to extract brain tissue from a dead animal such as a farm animal, most preferably a dead cow being assayed for the presence of prions. The device could readily be used for the extraction of brain tissue from any farm animal including cows, pigs, horses, sheep, goat or any animal including non-farm animals such as a deceased human or other primate.

Although the device of the invention could be produced in various sizes it is important that the size of the device be fairly close to the particularly preferred range as such allows for obtaining an adequate but not too large of a sample which can be readily assayed. Thus, the tube 3 preferably holds a volume of about 1 ml with the range preferably being 0.1 to 10 ml., more preferably 0.5 to 5 ml and most preferably graduated to show a measured volume of 1 ml with a very small (e.g. ±0.01 ml) volume of error. The inside diameter of the tube 3 should be approximately 3/16 of an inch (0.4 cm). The length of the tube “L” should be approximately three inches and the length “C” of the cuff 4 should be approximately 1/4 inch with about half of the cuff inserted into the tube 3 and half extending outside of the tube 3 with the cutting edge 13 of the cuff 4 extending outward.

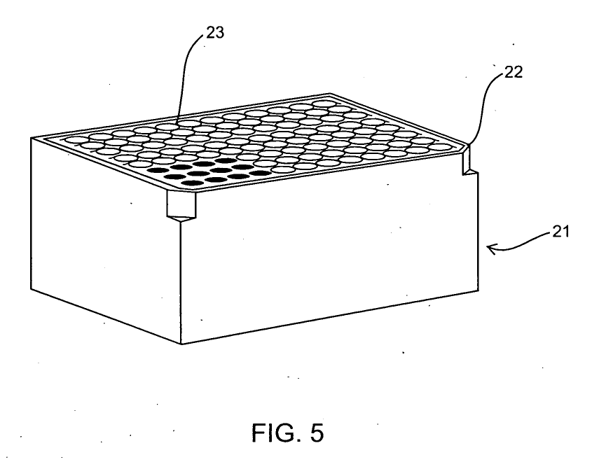

The system of the invention comprises the device 2 as shown in any of FIGS. 1-4 in combination with the 96 well plate 21 as shown in FIG. 5. The plate 21 is comprised of a plurality of wells and specifically includes 96 wells. The top 22 of the wells is coated with a breakable seal 23. The seal 23 may be comprised of any material and it has been found convenient to comprise the seal of a metal foil such as aluminum foil having no adhesive which adheres by pressure and conforming in shape to the top 22 surfaces to the wells.

The breakable seal 23 is punctured by the sharp end 13 of the cuff 4 by the device 2 shown in any of FIGS. 1-4. Once punctured the flange 11 of the plunger 8 is moved forward in order to expel the sample out of the tube 3 and into the well. By having the breakable seal 23 in place it is possible to easily determine that the well has been filled and keep the well sterile prior to breaking the seal. The next sample is then placed in the next available well for which the seal has not been broken. The process is repeated 96 times and with each tissue extraction a new device 2 is used. This avoids the necessity for sterilization of the device which would be necessary to avoid contamination from one sample to the next.

Each well of the 96 well plate preferably comprises a plurality of beads such as 2, 3, 4, 5 or 10 or more etc. small beads approximately 1 to 3 mm in diameter. The well may also have reagent materials such as proteinase K therein. Once all of the wells are filled the 96 well plate is shaken vigorously so that the beads aid in homogenizing the sample and mixing the reagent into the sample. When the sample has been sufficiently homogenized in all 96 wells the entire 96 well plate may be subjected to centrifugation. A more detailed description of the sample preparation is provided in the examples. After the sample is prepared the prepared sample is used in conducting an assay in order to determine if the sample is contaminated with prions. A more detailed description of the assay process is also provided in the examples.

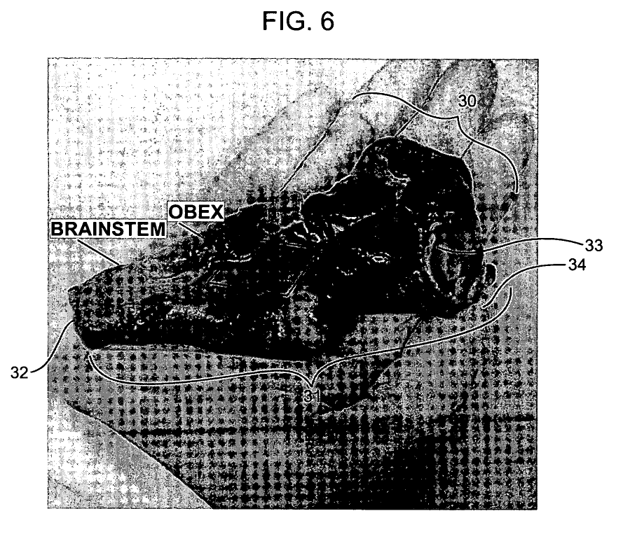

In order to use a device of the invention it is first important to extract the brainstem of a cow. Such a brainstem 31 has been extracted and is shown in the gloved hand 30 of a researcher in FIG. 6. FIG. 6 specifically identifies the obex region of the brainstem. When extracting the sample from the brainstem it is important to follow a certain procedure in order to extract tissue from the appropriate area and in order to minimize damage to any un-sampled section of the brainstem in that another section of the brainstem may be used for confirming a positive test. It is recommended that a user of the device extract away approximately ten samples in order to ensure that accurate and consistent tissue samples are obtained. It is desirable to obtain a tissue sample which as a weight in a range of about 0.25 to about 0.35 grams.

In order to extract the sample the brainstem 31 shown in FIG. 6 is placed in the palm of the user's gloved hand 30. To ensure additional protection the gloved hand may be placed inside a clear plastic bag. The sample is then oriented such that the spinal cord 32 in the hand 30 has its anterior portion 33 extending towards the thumb 34.

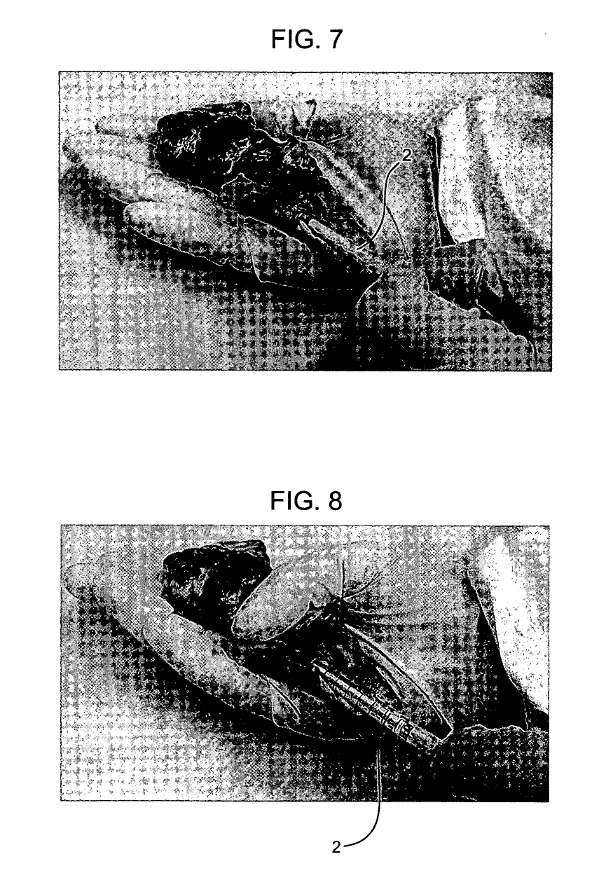

A sampling device 2 of a type as shown in FIG. 1 is then held at an angle of about 10 to 15° with respect to the sample as is shown within FIG. 7. The device 2 is inserted into the sample approximately 1 to 2 cm (0.5 to 0.75 inch) at an area posterior to the obex region which is shown in FIG. 6. As shown in FIG. 7 the device 2 is inserted into one side of the brainstem. Failure to limit the sampling to one side may make it difficult to check the results if a confirmation test is necessary. After inserting the tip of the device 2 into the brainstem as shown in FIG. 8 the user simultaneously begins to pull back, slowly, on the syringe decreasing the angle of the device relative to the sample slightly and continuing to push the tip of the device 2 into the obex region.

As shown in FIG. 8 the device is pushed into the center of the obex region while pulling back on the syringe. Once the device 2 has reached the center of the obex region the device should be twisted one complete turn and the tip of the device pushed against the wall of the obex in order to release or break the tissue sample away. The test sample is then drawn up into the syringe by the pressure differential by extracting the plunger. When the sample has broken away from the brainstem tissue the sample within the device 2 should be pulled further up into the syringe by withdrawing the plunger. After the sample is moved further up into the device 2 the device can be removed from the brainstem 31.

After the device 2 is taken out of the brainstem the plunger should be pushed forward slowly until the plunger contacts the tissue sample present within the device. At this point the user should confirm that the end or black piece of the plunger is positioned at 0.3 to 0.35 ml marks on the 1 ml device. If there is too much tissue in the device it is possible for the user should push out tissue into a waste container until a proper measurement is achieved. If there is too little tissue in the device then the sample material may be extruded into a weighing device and reinserted into the same side of the brainstem where the process can be repeated in order to extract additional material with the syringe.

The sample should be dispensed into a tubular opening 23 as shown within the 96 well plate 21 of FIG. 5. The sampling device 2 should then be discarded into a receptacle where all materials will be properly disposed of. It should be kept in mind that prions are infectious and that they are particularly difficult to inactivate. The brain stem sample should be returned to an appropriate container with appropriate marking indices in order to associate that brainstem with the sample deposited into the opening of the 96 well plate. By doing such it is possible to confirm a test particularly when a positive result is obtained. The process may be repeated multiple times until each of the openings within the 96 well plate is filled.

In general a sample is prepared by concentrating prions within the sample. This is carried out by homogenizing the sample and mixing the sample with a complexing agent which selectively complexes with prions within the sample. The complexing agent may be a compound such as sodium phosphotungstate. Those skilled in the art will recognize a range possible complexing agents which include other salts of phosphotunstic acid, antibodies, and any agent which selectively binds to prions forming a complex which has a higher specific gravity as compared to the prion by itself. The complexing agent is mixed with the sample for a time sufficient to form complexes between the agent and the prions which might be present in the sample. After the mixing has been completed and sufficient time and conditions have occurred the sample is subjected to centrifugation and the complexing agent aids in increasing the specific gravity by binding to the prions. Thereafter the sample may be treated with a proteinase which selectively degrades the other proteins present within the sample, i.e. proteins other than prions. The treatment process may be eliminated in some circumstances and when used may be with proteinase K or other suitable protease or other agent which degrades other proteins present in the sample e.g., heat, pressure or agitation. This treatment may be carried out prior to, during or after centrifugation. A more detailed description of sample preparation is provided within U.S. Pat. No. 5,977,324 issued Nov. 2, 1999 incorporated here by reference to disclose and describe a sample preparation method.

Once the sample has been properly prepared individual samples within the individuals wells are then subjected to an assay method in order to determine if the prepared sample is contaminated with prions. This is carried out by extracting the sample and dividing the sample into a first portion and a second portion. The first portion of the sample is contacted with a labeled antibody such as an antibody disclosed within U.S. Pat. No. 6,537,548 issued Mar. 25, 2003 incorporated herein by reference to disclose and describe antibodies, labeled antibodies, and methods of using such. The antibody binds to PrP proteins in their non-disease confirmation with a higher degree of affinity than the antibody binds to PrP protein and its disease conformation.

The second portion of the treated sample is then subjected to further treatment. This treatment causes PrP protein in the second disease related conformation (prions) to assume a different conformation which conformation has a higher degree of binding affinity for the labeled antibody as compared with its affinity for PrP protein in the second disease conformation (prions). Thereafter, the treated second portion is brought into contact with a labeled antibody of the same type used in contacting the first portion of the sample.

The level of binding of labeled antibody to PrP protein in the first portion is determined as is the level of binding to the treated PrP protein in the second sample. A comparison of the level of binding of the labeled antibody to PrP protein in the first portion of the sample is then compared with the level of binding in the second portion. Thereby it is possible to determine if the sample comprised PrP protein in the second disease conformation, i.e. if the sample included any prions. In essence the assay is carried out by relaxing the constricted conformation of prions so that they bind to the antibody. If there are no prions present then there will be no constricted conformation to relax. Those skilled in the art will understand that the treatment of the second portion may cause some increase in signal even if no prions are present. However, this increase can be accounted for and appropriate adjustments made so as to not assume a positive result when no prions are present. A more detailed description of such an assay is provided within U.S. Pat. No. 5,891,641 issued Apr. 6, 1999 incorporated herein to disclose and describe this and other assay methods. However, other assays for the detection of prions could be used to test the samples prepared in accordance with the present invention.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Examples 1-27

A device 2 as shown in FIG. 1 is used to extract a tissue sample from a cow brainstem in a manner as described above. 300 devices of the type shown in FIG. 1 were produced. In order to test the accuracy of the device and not test the samples 9 of those devices were used. The nine devices were in three categories with category 1 being the least sharp, category 2 being of medium sharpness and category 3 being the most sharp. Each device 2 as shown in FIG. 1 was used to extract 3 samples. This would not be done in practice in that a device would be discarded after extracting a single sample in order to avoid the possibility of contamination. The result of the 27 different tests are put forth below in terms of the weight of the samples obtained.

Weights (milligrams)

| | | | |
|---|---|---|---|
| 1 | 245 | 345 | 338 |
| 1 | 308 | 350 | 289 |
| 1 | 345 | 300 | 256 |
| 2 | 315 | 319 | 276 |
| 2 | 311 | 215 | 350 |
| 2 | 350 | 348 | 366 |
| 3 | 355 | 323 | 335 |
| 3 | 320 | 342 | 250 |
| 3 | 347 | 321 | 329 |

| | |
|---|---|
| Ave. | 316.5926 |
| Std. dev. | 38.63464 |
| % CV | 12.203327 |
| min | 215 |
| max | 366 |

As shown in the above examples the device 2 as shown in FIG. 1 makes it possible to repeatedly obtain substantially the same amount of brainstem samples. Those killed in the art will understand that it is somewhat difficult to obtain multiple samples from a cow brainstem and will appreciate the accuracy and convenience of the device described here.

### Example 28

A tissue extraction device 2 as shown in FIG. 1 is used to extract 300 milligrams of brain tissue from the brain of a slaughtered cow. The extracted 300 milligrams of tissue is placed in a well of a 96 well plate of the type as shown in FIG. 5. The well has therein a plurality of beads and proteinase K. After depositing the sample in the well the device 2 is discarded. Thereafter, a new device 2 is used to extract a sample from the brain of a different cow and the same process is repeated. The process is repeated 96 times until each well of the 96 well plate as shown in FIG. 2 has a sample of about 300 milligrams therein and 96 different devices 2 as shown in any of FIGS. 1-4 have been used and discarded. The 96 well plate is then shaken vigorously in order to homogenize the sample within each of the wells and intermix the proteinase K into the homogenized sample.

This example can also be carried out without the use of the 96 well plate. As indicated above it is possible to use individual tubes such as 2 ml tubes which may include beads into which the sample is placed and shaken in order to bring about homogenization of the brain tissue.

### Example 29

### Purification of Hamster PrP^{c} from Normal and PrP^{Sc} from Scrapie Infected Hamster Brains

The PrP^{c} protein can be purified as described in Pan, Stahl et al. (1992) Protein Sci 1:1343-1352; Pan, Baldwin et al. (1993) Proc Natl Acad Sci USA 90:10962-10966. Protein content can be determined by the BCA method. The purity of PrP^{c} protein, can be determined on SDS PAGE followed by silver staining and Western.

Standard Syrian hamster PrP^{Sc} can be purified from a standard pool of scrapie strain Sc237 infected hamster brains as described in Turk, Teplow et al. (1988) Eur J Biochem 176:21-30. The infectivity of this standard, as determined by an incubation time assay on Syrian hamsters after intracerebral inoculation, should be 10^{7.3} ID₅₀/ml and specific infectivity 10^{8.2} ID₅₀/mg of PrP^{Sc} protein. However, the specific infectivity may vary from lot to lot ±10^{0.5} ID₅₀/mg. The protein content can be determined by BCA assay using Bovine serum albumin as a standard. The preparation can be considered homogeneous with one major band on SDS PAGE after silver staining and Western Blots. The PrP proteins of the brain of other animals can be obtained in the same manner.

### Example 30

### Isolation of PrP^{Sc} from Bovine Brain

PrP^{Sc} is isolated from a fresh brain sample of a cow that exhibited symptoms of a neurological disorder consistent with the presence of PrP^{Sc}. Approximately 10 g of brain tissue is used to produce a homogenate. The brain tissue is flash-frozen in liquid nitrogen, and then homogenized using a standard mortar and pestle technique to dissociate the tissue for further extraction procedures. Phosphate buffered saline (PBS) pH 7.4 containing 4% (w/v) sodium dodecylsarcosinate (sarcosyl), an ionic surfactant, is added to the brain homogenate in a 1:5 (v/v) ratio to the brain homogenate. A solution of 4% sodium phosphotungstic acid (PTA) and 170 mM MgCl₂, pH 7.4, is added to the buffered homogenate solution to a final concentration of 0.2% PTA. The sample is exposed to the PTA for 16 hours at 30° C. on a rocking platform. At the end of 16 hours, Proteinase K is added to the solution providing a final concentration of 25 μg/ml, and the sample is incubated for one additional hour at 37° C. The addition of proteinase K generally increases the efficiency of the precipitation of PrP^{Sc}, in part by degrading other remaining proteins including PrP^{c}.

Following incubation, the sample is transferred to 1.5 ml sterile tubes, with approximately 1 ml aliquots of the PTA-homogenate solution per tube. The sample is centrifuged at 10,000 g in a table top centrifuge (Eppendorf) for 40 minutes at room temperature. The supernatant is decanted from the tubes, and each pellet is resuspended in sterile water to the desired overall protein concentration. Protease inhibitors are added to the solution: PMSF to a concentration 0.5 mM, Aprotinin to a final concentration of 2 mg/ml, and Leupeptin to a final concentration of 2 mg/ml. The protease inhibitors protect the sample from degradation under certain storage conditions. An aliquot of protein for current use is stored at 4° C. The remaining protein is aliquoted, and stored at −20° C.

The total protein content of the pellet is reduced 100 fold as compared to similar procedures known in the art. The PrP^{Sc} or PrP 27-30 content of this pellet represents approximately 40-60% of the total protein. This procedure thus results in a protein sample highly enriched in PrP^{Sc} protein species.

### Example 31

### Isolation of PrP^{Sc} from Human Brain

PrP^{Sc} is isolated from brain samples of a deceased individual suspected of being affected with a prion-based neurological disorder. Approximately 5 g of human brain tissue is used to produce a homogenate. The homogenate is produced using a dounce homogenizer to dissociated the tissue for protein extraction. A solution of 0.24 mM Triton-X, a non-ionic surfactant, in PBS pH 7.4 is added to a final 1:1 (v/v) ratio. A solution of 4% sodium phosphotungstic acid (PTA) and 170 mM MgCl₂, pH 7.4, is added to the buffered homogenate solution to a final concentration of 0.3% PTA. The sample is exposed to the PTA for 8 hours at 37° C. on a rocking platform.

Following incubation, the sample is transferred to 2.0 ml sterile tubes, with approximately 1 ml aliquots of the PTA-homogenate solution per tube. The sample is centrifuged at 14,000 g in a table top centrifuge (Brinkmann) for 30 minutes at room temperature. The supernatant is decanted from the tubes, and each pellet is resuspended in a 25 μg/ml proteinase K solution. The tubes are incubated for one additional hour, rocking, at 37° C. The sample is again centrifuged at 14,000 g in a table top centrifuge for 30 minutes at room temperature. The supernatant is decanted, and the sample resuspended in 100 μl of sterilized water. Concentration can be determined using spectrophotometric techniques. The PrP^{Sc} or PrP 27-30 content of this pellet represents approximately 40-60% of the total protein.

### Example 32

### Isolation of PrP^{Sc} from Sheep Brain

A brain sample from a sheep exhibiting neurological disorders is isolated and homogenized using a Polytron automated homogenizer. Approximately 1 gram of protein is homogenized in this fashion. PBS pH 7.4 containing 1% (w/v) sodium dodecyl sulfate (SDS) is added to the brain homogenate in a 5:1 (v/v) ratio. A solution of 4% sodium phosphotungstic acid (PTA) and 170 mM MgCl₂, pH 7.4, is added to the buffered homogenate solution to a final concentration of 0.25% PTA. The sample is exposed to the PTA for 1 hour at 45° C. on a rocking platform.

Following incubation, the sample is transferred to 1.5 ml sterile tubes, with approximately 1 ml aliquots of the PTA-homogenate solution per tube. The sample is centrifuged at 20,000 g in a table top centrifuge (Eppendorf) for 20 minutes at room temperature. The supernatant is decanted from the tubes, and each pellet is resuspended in sterile water to the desired overall protein concentration. An aliquot of protein for current use is stored at 4° C. The remaining protein is aliquoted, and stored at −20° C. The PrP^{Sc} or PrP 27-30 content of this pellet represents approximately 40-60% of the total protein.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A tissue extraction device, comprising:
a graduated, cylindrical, transparent tube comprising a first end and a second end connected to a tube flange;
a metal cuff inserted into the first end of the tube and extending outward from the first end, the cuff comprising a circular metal cylinder comprising a sharpened end extending from the tube; and
a plunger comprised of a shaft connected to a stopper at a first end and a plunger flange at a second end.

2. The device of claim 1, wherein the tube comprises an internal volume in the range of 0.1 ml to 10 ml.

3. The device of claim 1, wherein the cylinder comprises a volume in a range of 0.1 ml to 2 ml.

4. The device of claim 1, wherein the tube comprises a volume in a range of about 1 ml±5%.

5. The device of claim 1, further comprising:
a biasing means positioned between the tube flange and the plunger flange.

6. The device of claim 5, wherein the biasing means is chosen from a spring and an elastic polymeric material.

7. A tissue extraction system, comprising:
a tissue extraction device comprising a graduated, cylindrical, transparent tube comprising a first end and a second end, a metal cuff inserted into the first end of the tube and extending outward from the first end, the cuff comprising a circular metal cylinder comprising a sharpened end extending from the first end of the tube and a plunger comprised of a shaft connected to a stopper at a first end and a flange at a second end; and
a 96 well plate comprised of 96 interconnected wells having a continuous sealant cover over openings of all of the wells, wherein the internal diameter of each well of the 96 well plate is substantially the same as the external diameter of the cylindrical, transparent tube of the extraction device.

8. The system of claim 7, further comprising:
a plurality of beads in each of the wells.

9. The system of claim 7, further comprising:
a proteinase in each of the wells.

10. The system of claim 7, further comprising:
an agent which binds PrP protein in each of the wells.

11. The system of claim 8, further comprising:
proteinase K in each of the wells; and
a salt of phosphotungstic acid in each of the wells.

12. A method, comprising the steps of:
(a) inserting into a brain of a farm animal a tissue extraction device comprising a graduated, cylindrical, transparent tube comprising a first end and a second end, a metal cuff inserted into the first end of the tube and extending outward from the first end, the cuff comprising a circular metal cylinder comprising a sharpened end extending from the first end of the tube and a plunger comprised of a shaft connected to a stopper at a first end and a flange at a second end;
(b) withdrawing the plunger to draw about 0.3 milliliter of brain tissue into the tube; and
(c) moving the plunger forward to expel the brain tissue from the tube into a well of a 96 well plate comprised of 96 interconnected wells having a continuous sealant cover over openings of all of the wells, wherein the internal diameter of each well of the 96 well plate is substantially the same as the external diameter of the cylindrical, transparent tube of the extraction device which tube has therein a plurality of beads.

13. The method of claim 12, further comprising:
shaking the 96 well plate to homogenize the tissue in the well.

14. The method of claim 12, further comprising:
repeating steps (a), (b) and (c) whereby tissue from another animal is inserted into another tube in the repeated steps.

15. The method of claim 12, further comprising:
repeating steps (a), (b) and (c) a plurality if times wherein a new tissue extraction device is used in each set of repeated steps to extract tissue from another animal which is expelled into a different well.

16. The method of claim 15, wherein steps (a), (b) and (c) are carried out 96 times to expel tissue into all 96 wells.

17. The method of claim 16, further comprising:
shaking the 96 well plate to homogenize tissue in the wells.

18. The method of claim 17, further comprising:
centrifuging the 96 well plate.

19. A method, comprising the steps of:
(a) inserting into a brain of a farm animal a tissue extraction device comprising a graduated, cylindrical, transparent tube comprising a first end and a second end, a metal cuff inserted into the first end of the tube and extending outward from the first end, the cuff comprising a circular metal cylinder comprising a sharpened end extending from the first end of the tube and a plunger comprised of a shaft connected to a stopper at a first end and a flange at a second end;
(b) withdrawing the plunger to draw about 0.3 milliliter of brain tissue into the tube; and
(c) moving the plunger forward to expel the brain tissue from the tube into a container.

20. The method of claim 19, wherein the container is a cylindrical tube having a containment volume of approximately 2 ml and the container has therein a plurality of beads.
